(19) United States
(12) Patent Application Publication
(10) Pub. No.: US 2005/0245950 A1
(43) Pub. Date: Nov. 3, 2005

(54) Method and apparatus to mark an incision

(76) Inventor: Martin J. Kozlowski, Kutztown, PA (US)

Correspondence Address: Thomas W. Ryan;PIPER RUDNICK LLP, P.O. Box 64807, Chicago, IL, 60664-0807, US

(21) Appl. No.: 10/834,508

(22) Filed: Apr. 29, 2004

(51) Int. Cl. A61B 17/32

(52) U.S. Cl. 606/167

(57) ABSTRACT

An apparatus and method for precisely marking a surgical incision. A surgical knife contains a cutting blade which terminates proximally with a flat shoulder that is perpendicular to the cutting edge and contains a bioreactive stain dried thereon. In use, the surgical knife is inserted by the surgeon into the tissue of interest, up to the shoulder. The shoulder is then briefly placed against the incised tissue until the stain is rehydrated. The stain is then drawn from the knife to the tissue by capillary action, thus marking the site of the incision.

### BACKGROUND OF THE INVENTION

1. Technical Field of Invention

This invention relates to a method and apparatus for marking a surgical incision, and more particularly to a method and apparatus for providing a stain across a surgical incision made with a surgical knife.

2. Description of Prior Art

In many ophthalmologic surgeries, such as the removal of a cataract, one or more initial incisions are made in order to prepare the eye for additional incisions or the introduction of surgical instruments. Typically, such initial incisions are fairly small and difficult to see after they are made. Often, an incision is less than 2 mm in length. Also, such incisions may be obscured by the fluids and mucosa that naturally coat the surface of the eye. As a result, the surgeon must spend additional time keeping track of these incisions, locating them if temporarily lost or having to form a new incision or increasing the size of the initial incision. As can be appreciated, increased surgical time may lead to an increased chance of tissue morbidity, infection and longer patient recuperation.

It is known to use bioreactive stains to dye or highlight bodily tissues, such as ocular tissue. Such stains include, but are not limited to, Gentian violet, Indocyanine green, Methylene blue, Cresyl blue, VisionBlue and Trypan blue. Areas of interest are more easily seen upon application of the stain to the eye. While mostly effective, such staining methods suffer from several inherent problems when used in connection with surgical methods. First, the stain must be placed on the eye by the surgeon as a separate step either before or after the incision is made. While the stain can be placed in the area of interest prior to making an incision, this is generally undesirable. Also the apparatus for placing the stain on eye is yet another device that the surgeon must handle during the procedure. Most importantly, the stain probably will not be localized to the precise area of incision. Because of the properties of ocular stains, it is undesirable to use more than minute quantities. Therefore, there is a need for a method of placing a limited quantity of an ocular stain precisely by marking a surgical incision and an apparatus for accomplishing the same.

It is therefore an object of the present invention to provide a method and apparatus that overcomes the problems of the prior art.

It is another object of the present invention to provide an apparatus that is simple and easy to use by a surgeon and a method of accomplishing the same.

These and other objects of the invention will be apparent to those skilled in this art from the following detailed description of the preferred embodiments of the invention.

### SUMMARY OF INVENTION

The present invention provides an apparatus and method for precisely marking a surgical incision. The inventive surgical knife contains a cutting blade having a proximal end which terminates with a flat shoulder that is perpendicular to the cutting edge and contains a bioreactive stain dried thereon. In use, the inventive knife is inserted by the surgeon into the tissue of interest, up to the shoulder. The shoulder is then briefly placed against the incised tissue until the stain is rehydrated. The stain is drawn from the knife to the tissue by capillary action, thus marking the site of the incision.

### BRIEF DESCRIPTION OF THE DRAWINGS

Specific embodiments of the invention have been chosen for purposes of illustration and description, and are shown in the accompanying drawing, forming a part of the specification wherein:

FIG. 1 shows a perspective view of a marking knife according to the invention.

FIG. 2 illustrates a partial perspective view of the cutting portion of a marking knife according to the invention.

### DESCRIPTION OF PREFERRED EMBODIMENTS

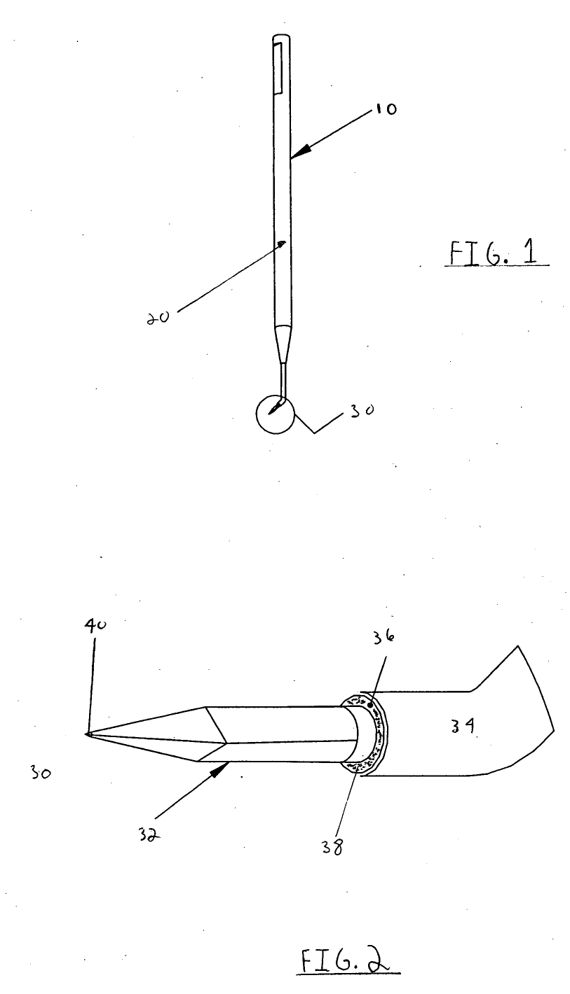

The structure of the invention described herein is best understood by reference to FIGS. 1 and 2. Referring to FIG. 1, the marking knife 10 of the instant invention is generally comprised of a handle 20 and a cutting portion 30. Referring now to FIG. 2, the cutting portion 30 is further comprised of a cutting blade 32 which is held in a retaining means 34, such as a tube, sheath or slot that may be integrally formed with the handle 20 or attached thereto as a separate structure. The blade 32 may be permanently mounted into the retaining means 34 or may be frictionally fit if the remainder of the knife 10 is intended for more than one use. One will also note in FIG. 2 that the retaining means 34 may be formed such that it presents the cutting blade 30 at an angle away from being perpendicular with the handle 20.

Blade 32 may be formed in any one of the conventional shapes known in the art such as a flat blade or a multi-surface blade (as is shown in FIG. 2) and may be curved or rounded. An important feature of the blade 32 is that the proximal end of the blade 32 terminates with a shoulder 36. In the preferred embodiment of the invention the shoulder is relatively flat and perpendicular to the cutting edge of the blade 32. It is contemplated that the shoulder 36 may have a different geometry, such as being convex or concave, depending on the intended use of the surgical knife 10. A bioreactive stain or dye 38 is placed onto the shoulder 36. In the preferred embodiment of the invention, the stain is placed on the shoulder 36 during manufacture and is either provided in a dry form dried directly on the shoulder 36 whereby the stain is otherwise stable until hydrated. Such bioreactive stains or dyes include, but are not limited to, Gentian violet, Indocyanine green, Methylene blue, Cresyl blue, VisionBlue and Trypan blue.

In an opthalmic surgery, the surgeon selects an appropriate site on the eye to make an incision with the marking knife 10 of the present invention. The tip 40 of the knife blade 32 is pressed into and through the outer surface of the eye (such as into the cornea in a cataract surgery), thus beginning the incision. The knife blade 32 is continued to be inserted into the eye until the shoulder 36 of the blade 32 touches the surface of the eye. The shoulder 36 is then held momentarily against the eye surface. The stain or dye 38 on the shoulder 36 is hydrated by the fluids and mucosa of the eye and the dye solution is then drawn to the incision by capillary action. After making a visual mark with the hydrated dye 38, the blade 32 is withdrawn and a visual mark remains on the eye for reference by the surgeon. It is contemplated that multiple incisions can be made and marked during a single surgical procedure. It should be noted that while the inventive marking knife and its method of use are described herein as being used in connection with ophthalmic surgeries, such uses should not be considered limited to this area. It is contemplated that the inventive marking knife and method can be used in other types of surgeries in which a stain 38 on the blade shoulder 36 is hydrated by an appropriate bodily fluid. The knife and method disclosed herein are equally adaptable for human and veterinary surgeries.

In addition to the structures, sequences, and uses immediately described above, it will be apparent to those skilled in the art that other modifications and variations can be made to the apparatus and method of the instant invention without diverging from the scope, spirit, or teaching of the invention. Therefore, it is the intention of the inventor that the description of instant invention should be considered illustrative and the invention is to be limited only as specified in the claims and equivalents thereto.

1. An surgical knife for forming and marking a surgical incision in a bodily tissue, said surgical knife comprising:
a) a handle;
b) a retaining means affixed to the handle; and
c) a cutting blade assembly affixed to said retaining means wherein said assembly is comprised of a cutting blade terminating in a proximal shoulder haveing a stain thereon,
whereby the cutting blade forms a surgical incision and marks said incision when the shoulder is placed against the bodily tissue and the stain is drawn thereto.

2. The surgical knife of claim 1 wherein the stain is a bioreactive dye.

3. The surgical knife of claim 2 wherein the bioreactive dye is selected from the group comprising Gentian violet, Indocyanine green, Methylene blue, Cresyl blue, VisionBlue and Trypan blue.

4. The surgical knife of claim 1 wherein the dye is affixed to the shoulder in a dried form, whereby the dye is rehydrated by a fluid on the bodily tissue and drawn thereto by capillary action.

5. The surgical knife of claim 1 wherein the cutting blade assembly is removably affixed to the retaining means, whereby a used cutting blade assembly may be removed and a new cutting blade assembly can be removably affixed such that the handle and retaining means are reusable.

6. The surgical knife of claim 1 wherein the shoulder is formed perpendicular to the cutting blade and relatively flat.

7. A method of forming and marking a surgical incision with a surgical knife comprising a handle affixed to a retaining means and a cutting blade assembly affixed to the retaining means and having a cutting blade terminating in a proximal shoulder having a stain affixed thereto, the method comprising:
a) selecting a site on a bodily tissue to make an surgical incision;
b) forming an incision in the bodily tissue at the selected site;
c) inserting the cutting blade into the bodily tissue up to the shoulder of the blade at the incision site;
d) contacting said blade shoulder on said bodily tissue; and
e) transferring the stain from the blade shoulder to said bodily tissue at said selected incision.

8. The method of claim 7 wherein the stain is a bioreactive dye is selected from the group comprising Gentian violet, Indocyanine green, Methylene blue, Cresyl blue, VisionBlue and Trypan blue.

9. The method of claim 7 wherein the bodily tissue is a human eye.

10. The method of claim 7 wherein the stain is transferred to the body tissue through capillary action.
